(19) United States
(12) Patent Application Publication
(10) Pub. No.: US 2013/0131446 A1
(43) Pub. Date: May 23, 2013

(54) ENDOSCOPIC OVERTUBE ASSEMBLY

(71) Applicants: Gerald W. Dryden, JR., Louisville, KY (US); Joseph Watson Vicars, III, Louisville, KY (US)

(72) Inventors: Gerald W. Dryden, JR., Louisville, KY (US); Joseph Watson Vicars, III, Louisville, KY (US)

(73) Assignee: UNIVERSITY OF LOUISVILLE RESEARCH FOUNDATION, INC., Louisville, KY (US)

(21) Appl. No.: 13/636,548

(22) PCT Filed: Mar. 23, 2011
(86) PCT No.: PCT/US11/29632

§ 371 Date: Nov. 14, 2012

Related U.S. Application Data
(60) Provisional application No. 61/316,700, filed on Mar. 23, 2010.

(51) Int. Cl. A61B 1/00; A61M 16/04; A61B 1/31; A61B 1/05; A61B 1/273; A61B 1/24; A61M 16/00

(52) U.S. Cl. CPC A61B 1/00154; A61B 1/00082; A61B 1/24; A61M 16/0497; A61M 16/0434; A61M 16/0057; A61B 1/00094; A61B 1/05; A61B 1/2733; A61B 1/2736; A61B 1/31
USPC 600/109; 600/115

(57) ABSTRACT

An endoscopic overtube assembly comprises: an inflatable overtube; and a subassembly configured for insertion into an orifice of a patient, said subassembly defining a central passageway, and said subassembly including an internal cassette for receiving and securing the inflatable overtube, wherein the internal cassette of said subassembly can be manipulated from a closed position to an open position in which the inflatable overtube can be readily extended and deployed from said subassembly. The inflatable overtube travels with the endoscopic device. Once appropriately positioned, the inflatable overtube is detached from the endoscopic device and inflated to become a substantially rigid conduit defining an instrument channel that provides ready access to a particular body cavity.

### CROSS-REFERENCE TO RELATED APPLICATIONS

The present application claims priority to U.S. Provisional Patent Application Ser. No. 61/316,700 filed on Mar. 23, 2010, the entire disclosure of which is incorporated herein by reference.

### BACKGROUND OF THE INVENTION

In performing endoscopic procedures, an overtube is often placed over the endoscopic device in order to provide a passageway for the endoscopic device as it is advanced into the esophagus/stomach (esophagogastroduodenoscopy), into the colon (colonoscopy), or into another body cavity. See, e.g., Communication from the ASGE Committee, “Overtube use in a gastrointestinal endoscopy,” Gastrointestinal Endoscopy, Vol. 70, No. 5: 828-34 (2009). However, existing overtubes suffer from various deficiencies. For instance, many overtubes are rigid and difficult to place. Pinch points can form between the endoscopic device and the overtube, which, in an esophagogastroduodenoscopy, can lead to esophageal perforations. Additionally, existing overtubes are of a fixed length and therefore are not suitable for certain applications.

### SUMMARY OF THE INVENTION

The present invention is an endoscopic overtube assembly that includes an inflatable overtube that can be extended to a desired length. In this regard, the inflatable overtube is extended in a deflated state as the endoscopic device to which it is attached is advanced into the esophagus/stomach (esophagogastroduodenoscopy), into the colon (colonoscopy), or into another body cavity. In other words, the inflatable overtube travels with the endoscopic device. Once appropriately positioned, the inflatable overtube is detached from the endoscopic device and inflated to become a substantially rigid conduit through which the endoscopic device can readily move. The inflatable overtube thus defines an instrument channel that provides ready access to a particular portion of the esophagus, stomach, or colon, while protecting the surrounding tissue during an endoscopy or similar procedure.

In one exemplary embodiment, the endoscopic overtube assembly is to be used to access the esophagus or stomach through the mouth of the patient. Accordingly, the endoscopic overtube assembly generally includes an inflatable overtube and a bite block subassembly. The bite block subassembly includes a bite block, a bite block sleeve, a locking ring, and an internal cassette for receiving and securing the inflatable overtube. The bite block is configured for insertion into the mouth of a patient. The bite block sleeve is then secured to the bite block. A central passageway passes through these two components, such that an endoscopic device can be passed through the bite block sleeve and the bite block. The internal cassette for receiving and securing the inflatable overtube is then received in the bite block sleeve. The internal cassette is not rigidly fixed to the bite block sleeve, but rather is adapted for movement relative to the bite block sleeve along a longitudinal axis of the bite block subassembly. The locking ring fits over and engages the upper edge of the bite block sleeve and controls the movement of the internal cassette relative to the bite block sleeve.

With respect to the movement of the internal cassette relative to the bite block sleeve, the internal cassette not only includes a cylindrical outer wall, but also includes a concentric, cylindrical inner wall that is positioned to fit in and engage a central channel defined by the bite block. The inflatable overtube is received in the annular cavity defined between the outer and inner walls of the internal cassette.

When the internal cassette is in a closed position, the cylindrical inner wall of the internal cassette fits in and engages the central channel defined by the bite block. Thus, the inflatable overtube is pinched between the cylindrical inner wall of the internal cassette and the bite block, thus preventing further deployment of the inflatable overtube through the bite block.

When the internal cassette is in an open position, the internal cassette is moved upward and away from the bite block, such that the inflatable overtube can be readily extended and deployed through the bite block.

In practice, the bite block is inserted into the mouth of a patient. The internal cassette is then moved upward and away from the bite block through manipulation of the locking ring. When in this open position, the inflatable overtube is free to move and extend through the bite block. The endoscopic device can then be advanced through the central passageway defined by the bite block subassembly. Since the camera end of the endoscopic device is attached to the distal end of the inflatable overtube, as the endoscopic device is advanced into the esophagus or stomach, the inflatable overtube travels with the endoscopic device. Once the inflatable overtube is appropriately positioned, the internal cassette is returned to the closed position with the cylindrical inner wall of the internal cassette fitting in and engaging the central channel defined by the bite block, thus preventing further deployment of the inflatable overtube. The inflatable overtube is detached from the endoscopic device and inflated to become a substantially rigid conduit.

Although the endoscopic overtube assembly of the present invention may be particularly well suited for an esophagogastroduodenoscopy, it may also be used in other procedures and in other body cavities. For example, the endoscopic overtube assembly of the present invention could be used in a colonoscopy. In such use, the endoscopic overtube assembly would function the same way as in a esophagogastroduodenoscopy, but the “bite block” element would be configured for insertion into and engagement with the anus as opposed to the mouth. For another example, the endoscopic overtube assembly of the present invention may also be used in Natural Orifice Transluminal Endoscopic Surgery (NOTES) for maintaining access to the intra-abdominal space.

### DESCRIPTION OF THE DRAWINGS

FIG. 1 is a perspective view of an exemplary endoscopic overtube assembly made in accordance with the present invention;

FIG. 2 is a side view of the exemplary endoscopic overtube assembly of FIG. 1;

FIG. 3 is a top view of the exemplary endoscopic overtube assembly of FIG. 1;

FIG. 4 is a bottom view of the exemplary endoscopic overtube assembly of FIG. 1;

FIG. 5 is a side view of the exemplary endoscopic overtube assembly of FIG. 1, but wherein the bite block sleeve and the locking ring have been removed for purposes of illustration;

FIG. 6 is a perspective view of the locking ring of the exemplary endoscopic overtube assembly of FIG. 1;

FIG. 7A is a sectional view of the exemplary endoscopic overtube assembly of FIG. 1, wherein the internal cassette is in a closed position with the cylindrical inner wall of the internal cassette fitting in and engaging the central channel defined by the bite block;

FIG. 7B is a sectional view of the exemplary endoscopic overtube assembly of FIG. 1 similar to FIG. 7A, but wherein the internal cassette is in an open position; and

FIG. 8 is a perspective view of the distal end of the overtube of the exemplary endoscopic overtube assembly of FIG. 1, illustrating its connection to an endoscopic device.

### DETAILED DESCRIPTION OF THE INVENTION

The present invention is an endoscopic overtube assembly that includes an inflatable overtube that can be extended to a desired length. In this regard, the inflatable overtube is extended in a deflated state as the endoscopic device to which it is attached is advanced into the esophagus/stomach (esophagogastroduodenoscopy), into the colon (colonoscopy), or into another body cavity. In other words, the inflatable overtube travels with the endoscopic device. Once appropriately positioned, the inflatable overtube is detached from the endoscopic device and inflated to become a substantially rigid conduit through which the endoscopic device can readily move. The inflatable overtube thus defines an instrument channel that provides ready access to a particular portion of the esophagus, stomach, or colon, while protecting the surrounding tissue during an endoscopy or similar procedure.

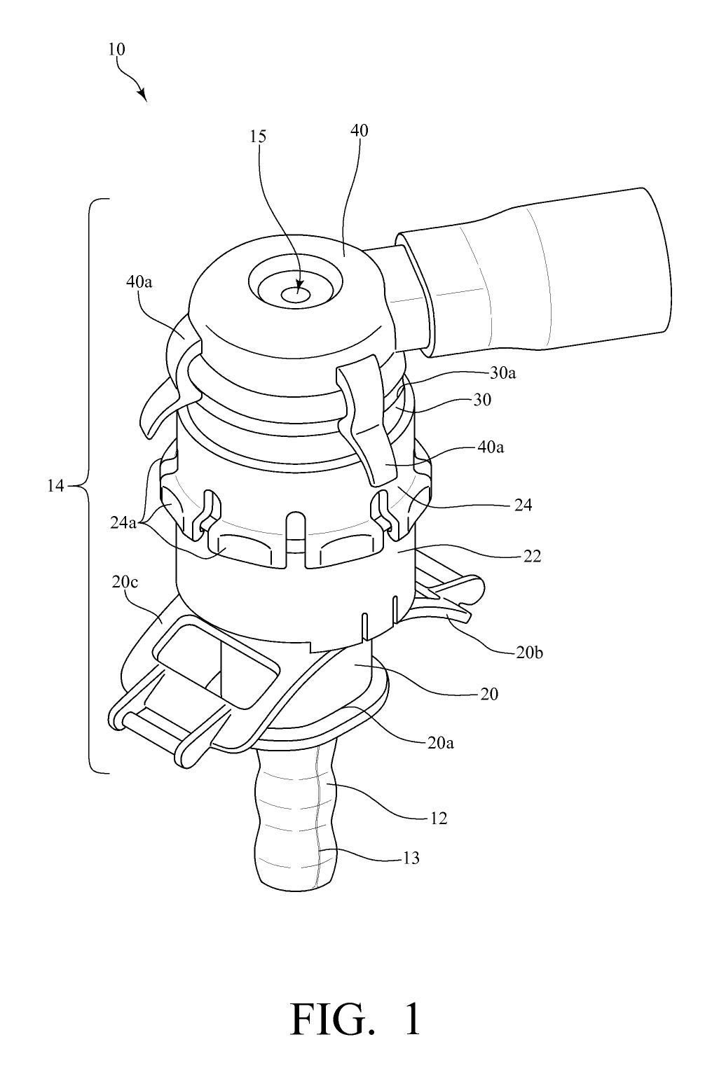

FIG. 1 is a perspective view of an exemplary endoscopic overtube assembly 10 made in accordance with the present invention. In this exemplary embodiment, the endoscopic overtube assembly 10 is to be used to access the esophagus or stomach through the mouth of the patient. Accordingly, the endoscopic overtube assembly 10 generally includes an inflatable overtube 12 (which is partially deployed in FIG. 1) and a bite block subassembly 14. The bite block subassembly 14 includes a bite block 20, a bite block sleeve 22, a locking ring 24, and an internal cassette 30 for receiving and securing the inflatable overtube 12. A central passageway 15 is also defined by the bite block subassembly 14 through the various components. In this exemplary embodiment, the bite block subassembly 14 further includes a suction adapter/port 40, the function of which is further described below.

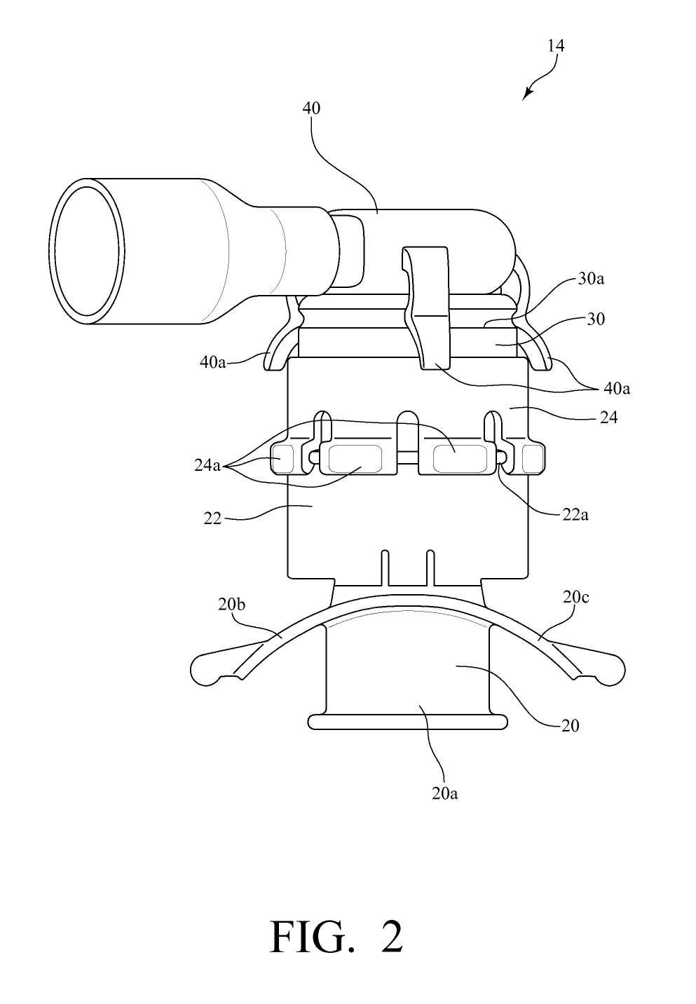

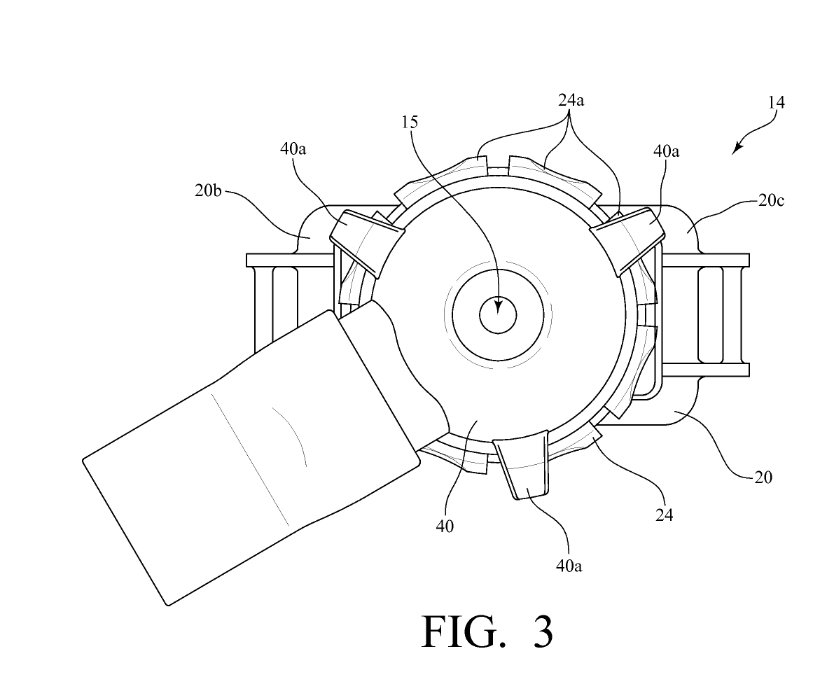

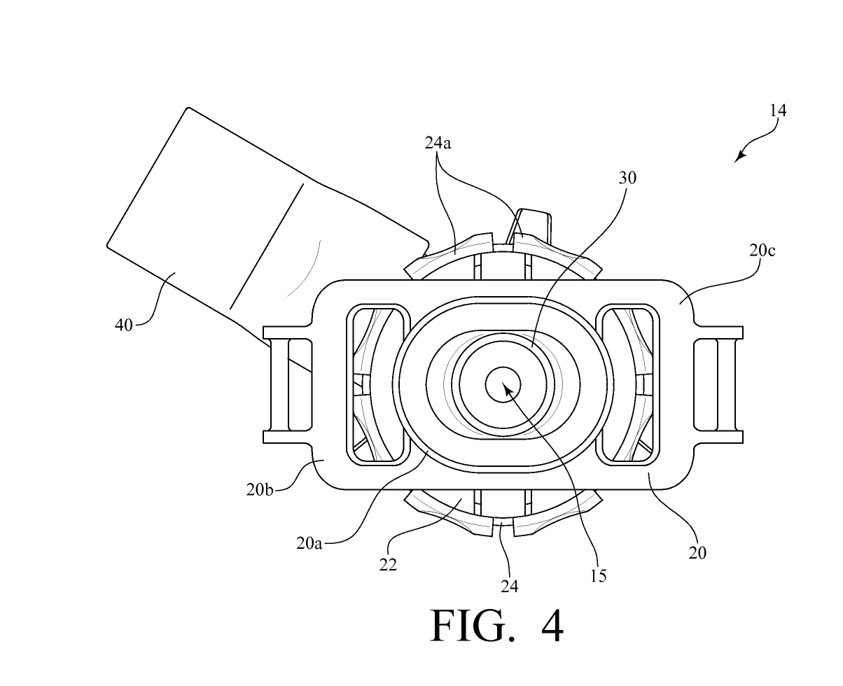

FIGS. 2-4 are various views of the bite block subassembly 14 of the exemplary endoscopic overtube assembly 10. Specifically, and as shown in FIGS. 2-4, the bite block 20 is configured for insertion into the mouth of a patient, having a center portion 20a that extends into the mouth of the patient and wing portions 20b, 20c that extend across and rest against the face of the patient. The bite block sleeve 22 is then secured to the bite block 20. The central passageway 15 referenced above passes through these two components, such that an endoscopic device can be passed through the bite block sleeve 22 and the bite block 20, as further described below.

The internal cassette 30 for receiving and securing the inflatable overtube 12 is then received in the bite block sleeve 22. The internal cassette 30 is not rigidly fixed to the bite block sleeve 22, but rather is adapted for movement relative to the bite block sleeve 22 along a longitudinal axis of the bite block subassembly 14, i.e., up and down within the bite block sleeve 22. The locking ring 24 then fits over and engages the upper edge of the bite block sleeve 22 and controls the movement of the internal cassette 30 relative to the bite block sleeve 22, as further described below. With respect to the engagement of the locking ring 24 to the bite block sleeve 22, in this exemplary embodiment, the locking ring 24 includes multiple integral tabs 24a that are snap fit over a circumferential flange 22a along the upper edge of the bite block sleeve 22. Thus, while connected to the bite block sleeve 22, the locking ring 24 can still rotate relative to the bite block sleeve 22.

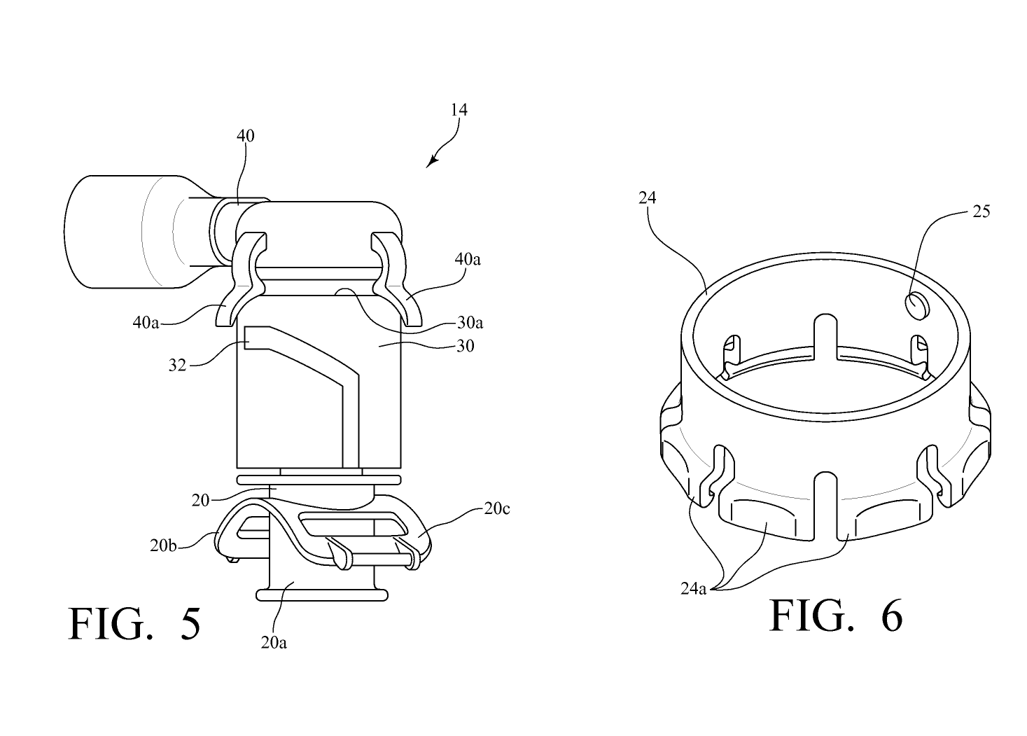

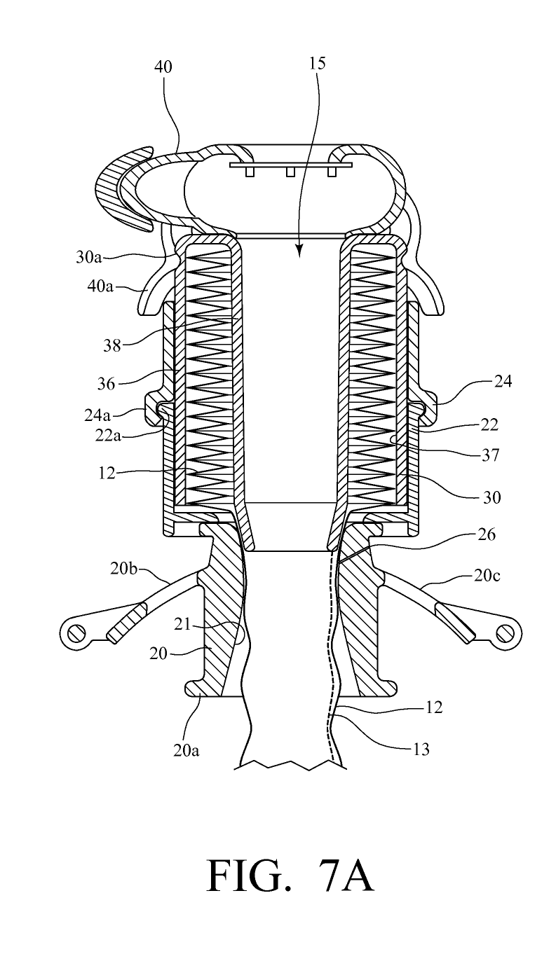

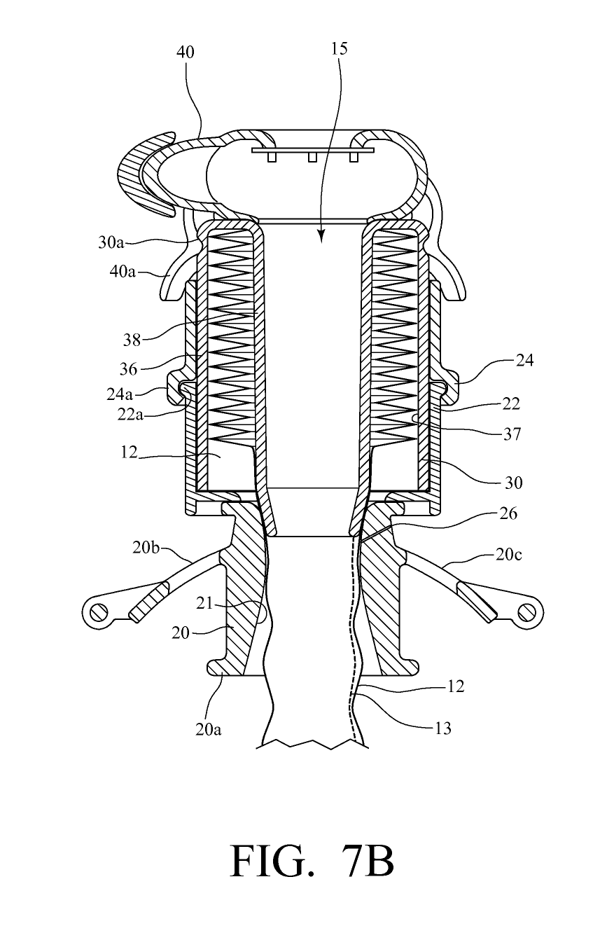

Referring now to FIG. 5, in which the bite block sleeve 22 and the locking ring 24 have been removed for purposes of illustration, there is a groove 32 in the exterior surface of the internal cassette 30 on one side of the internal cassette 30, and there is also a second identical groove (not shown) on the opposite side of the internal cassette 30. Referring now to FIG. 6, which is a perspective view of the locking ring 24 itself, the locking ring 24 is provided with two bosses 25 (one of which is shown in FIG. 7) extending from its internal surface that are received in the grooves 32 defined in the exterior surface of the internal cassette 30. Thus, the locking ring 24 controls and limits the movement of the internal cassette 30 relative to the bite block sleeve 22, as the bosses 25 ride in the grooves 32 defined in the exterior surface of the internal cassette 30. Specifically, as the locking ring 24 is rotated in one direction relative to the bite block sleeve 22, the internal cassette 30 is moved upward by the engagement of the bosses 25 in the grooves 32. When the locking ring 24 is rotated in the opposite direction, the internal cassette 30 is moved downward.

With respect to the movement of the internal cassette 30 relative to the bite block sleeve 22, reference is now made to the sectional views of FIGS. 7A and 7B. First, as shown in FIGS. 7A and 7B, the internal cassette 30 not only includes a cylindrical outer wall 36, but also includes a concentric, cylindrical inner wall 38 that is positioned to fit in and engage a central channel 21 defined by the bite block 20. This central channel 21 is part of the overall central passageway 15 defined by the bite block subassembly 14. Thus, as shown in FIGS. 7A and 7B, the inflatable overtube 12 is received in the annular cavity 37 defined between the outer and inner walls 36, 38 of the internal cassette 30.

In FIG. 7A, the internal cassette 30 is in a closed position, with the cylindrical inner wall 38 of the internal cassette 30 fitting in and engaging the central channel 21 defined by the bite block 20. Thus, as shown in FIG. 7A, the inflatable overtube 12 is pinched between the cylindrical inner wall 38 of the internal cassette 30 and the bite block 20, thus preventing further deployment of the inflatable overtube 12 through the bite block 20. Furthermore, and referring back to FIGS. 5 and 6, in this closed position, the respective bosses 25 of the locking ring 24 would be positioned at the upper distal ends 32a of the grooves 32.

In FIG. 7B, the internal cassette 30 is in an open position. Specifically, the internal cassette 30 has been moved upward and away from the bite block 20, again with the bosses 25 extending from the internal surfaces of the locking ring 24 riding in the grooves 32 defined in the exterior surface of the internal cassette 30. When in this open position, the inflatable overtube can be readily extended and deployed through the bite block 20.

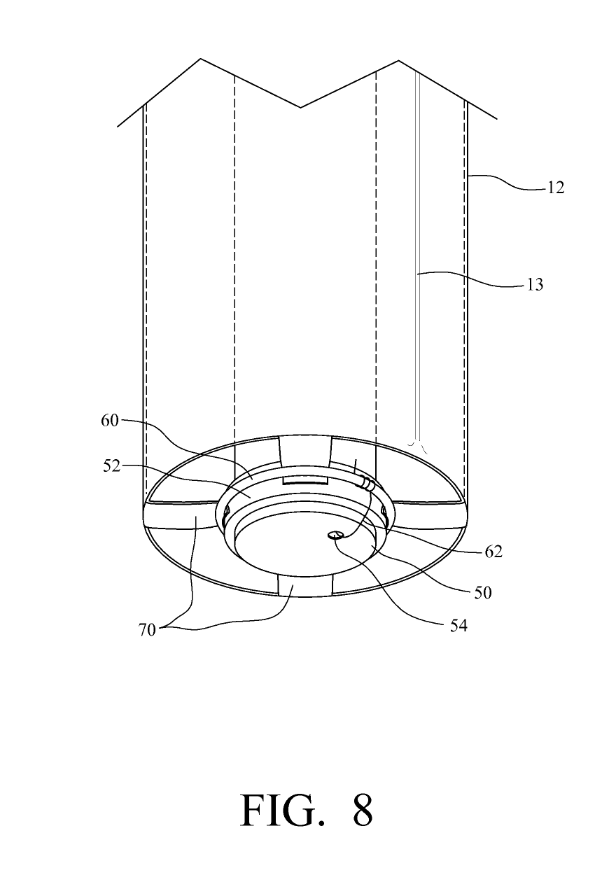

In practice, the bite block 20 is inserted into the mouth of a patient. The internal cassette 30 is then moved upward and away from the bite block 20 through manipulation of the locking ring 24. When in this open position, the inflatable overtube 12 is free to move and extend through the bite block 20. The endoscopic device 50 (as shown in FIG. 8) can then be advanced through the central passageway 15 defined by the bite block subassembly 14. The camera end of the endoscopic device 50 is attached to the distal end of the inflatable overtube 12, as further described below. Thus, as the endoscopic device 50 is advanced into the esophagus or stomach, the inflatable overtube 12 travels with the endoscopic device 50. Furthermore, the camera can be activated during such advancement to visualize the positioning of the inflatable overtube 12 and the endoscopic device 50. Once the inflatable overtube 12 is appropriately positioned, the internal cassette 30 is returned to the closed position with the cylindrical inner wall 38 of the internal cassette 30 fitting in and engaging the central channel 21 defined by the bite block 20, thus preventing further deployment of the inflatable overtube 12. The inflatable overtube 12 is detached from the endoscopic device 50 and inflated to become a substantially rigid conduit through which the endoscopic device 50 can readily move. The inflatable overtube 12 thus effectively serves as an instrument channel that provides ready access to a particular portion of the esophagus or stomach while protecting the surrounding tissue during an endoscopy or similar procedure.

With respect to the attachment of the endoscopic device 50 to the distal end of the inflatable overtube 12, along with the subsequent detachment of the inflatable overtube 12 from the endoscopic device 50 once positioned for use, various means of attachment could be employed without departing from the spirit and scope of the present invention. In one exemplary embodiment, and as shown in FIG. 8, an elastomeric ring 60 (or O-ring) fits over and around an annular cap 52 that is attached to the camera end of the endoscopic device 50. The elastomeric ring 60 pinches tabs 70 extending from the inflatable overtube 12 between the elastomeric ring 60 and the annular cap 52. Once the inflatable overtube 12 has been appropriately positioned, a trip wire 62 is pulled. The trip wire 62 extends through an instrument channel 54 defined by the endoscopic device 50 and is secured to the elastomeric ring 60. By pulling the trip wire 62, the elastomeric ring 60 is pulled off of the endoscopic device 50, releasing the tabs 70 and disengaging the endoscopic device 50 from the inflatable overtube 12.

With respect to the inflation of the inflatable overtube 12, this can also be achieved using various means without departing from the spirit and scope of the present invention. In one exemplary embodiment, and as also shown in FIG. 8, the inflatable overtube has a simple double-walled construction (resulting in a substantially annular cross-section). To inflate the overtube 12, air is introduced into the interior volume between the inner and outer walls of the inflatable overtube 12. In this regard, since the inflatable overtube 12 would only be extended to a length that is appropriate and necessary for a given procedure, it would be preferable to introduce air into the interior volume between the inner and outer walls of the inflatable overtube 12 from the distal end, so that the inflatable overtube 12 inflates from the distal end back to the point where the inflatable overtube 12 is pinched between the cylindrical inner wall 38 of the internal cassette 30 and the bite block 20, as described above. Thus, in this exemplary embodiment, the inflatable overtube 12 includes an air delivery tube 13 that runs along the outside surface of the inflatable overtube 12 and into the bite block subassembly 14 for connection to an air supply for inflation.

With respect to the connection of the air delivery tube 13 of the inflatable overtube 12, this can also be achieved using various means without departing from the spirit and scope of the present invention. In FIGS. 7A and 7B, there is a port 26 defined through the bite block 20 that allows access to the air delivery tube 13 of the inflatable overtube 12. An inflation needle can then be inserted into the port 26 to pierce and deliver air into the air delivery tube 13.

In an alternative embodiment, instead of a simple double-walled construction, an inflatable overtube for use with the endoscopic overtube assembly of the present invention could have a double-walled construction, but then include patterns of welds that connect the inner and outer walls of the inflatable overtube at selection locations. Such patterning can be used to limit and/or control expansion of the inflatable overtube.

In yet another alternative embodiment, the inflatable overtube could be constructed with a mesh or gridwork of interconnected air passageways in a manner similar to that described in U.S. Pat. No. 6,293,968 entitled “Inflatable Intraluminal Vascular Stent.”

Finally, with respect to possible constructions of an inflatable overtube for use with the endoscopic overtube assembly of the present invention, it is contemplated that the inflatable overtube could be provided with a flared or “bell shape” at its distal end, which could serve as a flange to assist the removal of foreign bodies and could also serve as a barrier for regurgitation of esophageal and gastric contents.

Returning now to FIGS. 1-3, as a further refinement, in this exemplary embodiment, the bite block subassembly 14 includes a suction adapter/port 40. The suction adapter/port 40 is secured to the distal end of the internal cassette 30. Specifically, the suction adapter/port 40 includes multiple integral fingers 40a that extend downwardly and engage a circumferential groove 30a defined near the upper edge of the internal cassette 30. The suction adapter/port 40 is in fluid communication with the central passageway 15 defined by the bite block subassembly 14 and the central volume of the inflatable overtube 12, thus allowing the inflatable overtube 12 to also serve as a conduit for suctioning large particulate matter from the stomach in the event of gastrointestinal bleeding or retention of food, in lieu of current nasogastric tubes or aspirator kits.

Finally, although the endoscopic overtube assembly of the present invention may be particularly well suited for an esophagogastroduodenoscopy as described above with respect to FIGS. 1-8, it may also be used in other procedures and in other body cavities. For instance, as mentioned above, the endoscopic overtube assembly of the present invention could be used in a colonoscopy. It would function the same way as in a esophagogastroduodenoscopy, but the “bite block” element would be configured for insertion into and engagement with the anus as opposed to the mouth. For another example, Natural Orifice Transluminal Endoscopic Surgery (NOTES) is an emerging technology that allows a surgeon to insert surgical instruments through a natural orifice (such as the mouth, anus, or vagina) and then access the abdominal cavity through a defect created in the stomach, colon, bladder or vagina, thus avoiding any external incisions or scars. Because the inflatable overtube of the present invention can be readily deployed with different lengths, it may also be useful in a NOTES procedure for maintaining access to the intra-abdominal space. In such a procedure, the inflatable overtube would also provide a channel through which air or carbon dioxide can be passed into the abdominal cavity.

One of ordinary skill in the art will recognize that additional embodiments are possible without departing from the teachings of the present invention or the scope of the claims which follow. This detailed description, and particularly the specific details of the exemplary embodiments disclosed herein, is given primarily for clarity of understanding, and no unnecessary limitations are to be understood therefrom, for modifications will become obvious to those skilled in the art upon reading this disclosure and may be made without departing from the spirit or scope of the claimed invention.

What is claimed is:

1. An endoscopic overtube assembly, comprising:
an inflatable overtube; and
a bite block subassembly adapted for insertion into a mouth of a patient, said bite block subassembly defining a central passageway allowing for passage of a endoscopic device through said bite block subassembly, and said bite block subassembly including an internal cassette for receiving and securing the inflatable overtube;
wherein the internal cassette of said bite block subassembly can be manipulated from a closed position to an open position in which the inflatable overtube can be readily extended and deployed from said bite block subassembly.

2. The endoscopic overtube assembly as recited in claim 1, in which said bite block subassembly further includes:
a bite block for insertion into the mouth of the patient;
a bite block sleeve secured to the bite block, with the internal cassette received in and adapted for movement relative to the bite block sleeve along a longitudinal axis of said bite block subassembly; and
a locking ring fitting over and engaging an upper edge of the bite block sleeve and controlling the movement of the internal cassette relative to the bite block sleeve, such that the internal cassette of said bite block subassembly can be manipulated from the closed position to the open position.

3. The endoscopic overtube assembly as recited in claim 2, in which the bite block includes a center portion that extends into the mouth of the patient and wing portions that extend across and rest against a face of the patient.

4. The endoscopic overtube assembly as recited in claim 2, wherein the locking ring includes multiple integral tabs that are snap fit over a circumferential flange along the upper edge of the bite block sleeve, such that the locking ring can rotate relative to the bite block sleeve.

5. The endoscopic overtube assembly as recited in claim 4, wherein one or more grooves are defined in an exterior surface of the internal cassette, and wherein the locking ring includes one or more bosses extending from its internal surface that are received in the one or more grooves defined in the exterior surface of the internal cassette, such that rotation of the locking ring in one direction relative to the bite block sleeve moves the internal cassette upward, while rotation of the locking ring in an opposite direction moves the internal cassette downward.

6. The endoscopic overtube assembly as recited in claim 2, wherein the internal cassette includes an outer wall and an inner wall, and wherein the inflatable overtube is received and secured in an annular cavity defined between the outer wall and the inner wall of the internal cassette.

7. The endoscopic overtube assembly as recited in claim 6, wherein, in the closed position, the inner wall of the internal cassette fits in and engages a central channel defined by the bite block, such that the inflatable overtube is pinched between the inner wall of the internal cassette and the bite block, thus preventing extension or deployment of the inflatable overtube through the bite block.

8. The endoscopic overtube assembly as recited in claim 5, wherein the internal cassette includes an outer wall and an inner wall, and wherein the inflatable overtube is received and secured in an annular cavity defined between the outer wall and the inner wall of the internal cassette.

9. The endoscopic overtube assembly as recited in claim 8, wherein, in the closed position, as a result of a rotation the locking ring in one direction relative to the bite block sleeve, the internal cassette is positioned such that the inner wall of the internal cassette fits in and engages a central channel defined by the bite block, such that the inflatable overtube is pinched between the inner wall of the internal cassette and the bite block, thus preventing extension or deployment of the inflatable overtube through the bite block.

10. The endoscopic overtube assembly as recited in claim 1, and further comprising an air delivery tube extending along an outside surface of the inflatable overtube and adapted for connecting the inflatable overtube to an air supply.

11. The endoscopic overtube assembly as recited in claim 1, and further comprising a suction adapter/port secured to the internal cassette and in fluid communication with the central passageway defined by said bite block subassembly.

12. An endoscopic overtube assembly, comprising:
an inflatable overtube; and
a subassembly configured for insertion into an orifice of a patient, said subassembly defining a central passageway, and said subassembly including an internal cassette for receiving and securing the inflatable overtube;
wherein the internal cassette of said subassembly can be manipulated from a closed position to an open position in which the inflatable overtube can be readily extended and deployed from said subassembly.

13. The endoscopic overtube assembly as recited in claim 12, wherein the internal cassette includes an outer wall and an inner wall, and wherein the inflatable overtube is received and secured in an annular cavity defined between the outer wall and the inner wall of the internal cassette.

14. The endoscopic overtube assembly as recited in claim 13, and further comprising an air delivery tube extending along an outside surface of the inflatable overtube and adapted for connecting the inflatable overtube to an air supply.

15. A combination, comprising:
an endoscopic device; and
an endoscopic overtube assembly including an inflatable overtube, a distal end of the inflatable overtube attached to a camera end of the endoscopic device, such that, as the endoscopic device is advanced into a body cavity, the inflatable overtube travels with the endoscopic device and is advanced into the body cavity.

16. The combination as recited in claim 15, and further comprising a ring that fits over and around the camera end of the endoscopic device, said ring engaging one or more tabs extending from the inflatable overtube.

17. The combination as recited in claim 16, and further comprising a trip wire extending through an instrument channel defined by the endoscopic device and secured to the ring, such that, by pulling the trip wire, the ring is pulled off of the endoscopic device, thus releasing the one or more tabs and disengaging the endoscopic device from the inflatable overtube.

18. The combination as recited in claim 15, wherein said endoscopic overtube assembly further comprises:
a subassembly configured for insertion into an orifice of a patient, said subassembly defining a central passageway, and said subassembly including an internal cassette for receiving and securing the inflatable overtube;
wherein the internal cassette of said subassembly can be manipulated from a closed position to an open position in which the inflatable overtube can be readily extended and deployed from said subassembly.

19. The endoscopic overtube assembly as recited in claim 18, wherein the internal cassette includes an outer wall and an inner wall, and wherein said inflatable overtube is received and secured in an annular cavity defined between the outer wall and the inner wall of the internal cassette.

20. The combination as recited in claim 18, wherein said endoscopic overtube assembly further comprises:
a bite block for insertion into a mouth of the patient;
a bite block sleeve secured to the bite block, with the internal cassette received in and adapted for movement relative to the bite block sleeve along the longitudinal axis of said subassembly; and
a locking ring fitting over and engaging an upper edge of the bite block sleeve and controlling the movement of the internal cassette relative to the bite block sleeve, such that the internal cassette of said subassembly can be manipulated from the closed position to the open position.

21. The endoscopic overtube assembly as recited in claim 20, wherein the locking ring includes multiple integral tabs that are snap fit over a circumferential flange along the upper edge of the bite block sleeve, such that locking ring can rotate relative to the bite block sleeve.

22. The endoscopic overtube assembly as recited in claim 20, wherein one or more grooves are defined in an exterior surface of the internal cassette, and wherein said locking ring includes one or more bosses extending from its internal surface that are received in the one or more grooves defined in the exterior surface of the internal cassette, such that rotation of the locking ring in one direction relative to the bite block sleeve moves the internal cassette upward, while rotation of the locking ring in an opposite direction moves the internal cassette downward.

23. The endoscopic overtube assembly as recited in claim 22, wherein, in the closed position, an inner wall of the internal cassette fits in and engages a central channel defined by the bite block, such that the inflatable overtube is pinched between the inner wall of the internal cassette and the bite block, thus preventing extension or deployment of the inflatable overtube through the bite block.

24. The endoscopic overtube assembly as recited in claim 18, and further comprising a suction adapter/port secured the internal cassette and in fluid communication with the central passageway defined by said subassembly.
